(19) United States
(12) Patent Application Publication
(10) Pub. No.: US 2016/0338669 A1
(43) Pub. Date: Nov. 24, 2016

(54) ULTRASONIC PROBE AND INJECTION MOLDING METHOD FOR SAME

(71) Applicant: NIHON DEMPA KOGYO CO., LTD., Tokyo (JP)

(72) Inventor: YOJI NAKA, SAITAMA (JP)

(73) Assignee: NIHON DEMPA KOGYO CO., LTD., Tokyo (JP)

(21) Appl. No.: 15/110,944

(22) PCT Filed: May 13, 2015
(86) PCT No.: PCT/JP2015/063760

§ 371 Date: Jul. 12, 2016

(30) Foreign Application Priority Data
May 16, 2014 (JP) 2014-101990

(51) Int. Cl. A61B 8/00; B29C 45/00

(52) U.S. Cl. CPC A61B 8/4444; A61B 8/4272; B29C 45/0025; B29C 2045/0027; B29K 2101/12

(57) ABSTRACT

An ultrasonic probe is provided and includes: an ultrasonic transmission and reception unit, being provided inside a housing; an acoustic transmission medium, being sealed in the housing; and a drive device for oscillating the ultrasonic transmission and reception unit. The housing is an injection-molded article by plastic material, and has a container shape with an opening in one direction. The container shape includes: a bottom surface portion through which ultrasonic waves transmit, and a peripheral portion being engaged with a probe body. The housing is injection-molded by providing the peripheral portion having a thickness thicker than a thickness of the bottom surface portion, providing a vertical groove parallel to a direction of mold release at a part of the peripheral portion; and providing a gate for flowing a molten resin at the peripheral portion apart from the vertical groove.

### TECHNICAL FIELD

The present invention relates to an ultrasonic probe and an injection molding method for the same, and more particularly relates to an ultrasonic probe and an injection molding method for the same in which a vertical groove is formed on a periphery portion of a housing of the ultrasonic probe which is formed by injection molding so as to control weld line to be formed at a thick periphery portion of the housing during injection molding.

### BACKGROUND ART

Conventionally, for example, a short-axis oscillating ultrasonic probe 1 for medical diagnosis, as shown in FIG. 4 and FIG. 5, comprises a housing 2 made of plastic material, a grip case 3 engaging with the housing 2, and a power supply cable 6 for supplying power to a drive device for oscillating a probe body 4. And, the probe body 4 constituting an ultrasonic transmission and reception unit including a piezoelectric element group and others is disposed at a base 5 provided inside the housing 2, and an acoustic propagation medium L, e.g., oil is also contained and sealed in the housing 2, and a body contacting surface S of the housing 2 is brought into contact with a body surface of a patient and the probe body 4 is oscillated in a short-axial direction of the piezoelectric element group, so as to take in three dimensional data.

The housing of this type of ultrasonic probe, as well as the linear oscillating type ultrasonic probe which comprises a drive device for oscillating the probe body linearly in the longitudinal direction, is manufactured by injecting plastic material into a mold.

Here, in such a housing, a portion of the bottom surface or the like which transmits ultrasonic waves, requires uniform thickness and finish since it is directly in contact with a body surface of a patient for ultrasonic transmission. Therefore, for the injection molding of the housing, typically, a gate that is an inlet of molten resin is located on a thick peripheral portion of the housing, avoiding the thin portion of the bottom surface.

For example, as shown in FIG. 6, (for explanation, shown in upside down of FIG. 5), the housing 2 has an outer shape like a rugby ball cut into two along a center line in a longitudinal direction, is manufactured by injecting plastic material into a mold, and comprises a peripheral portion 21 which is thick and extends in a vertical direction from an engaging surface 25 to be engaged with the grip case 3 of the ultrasonic probe, and a bottom surface portion 22 which is thin and has a curved surface shape so as to cover the peripheral portion 21. Then, mounting pins 23, for example, four, are made upright integrated with an inner bottom surface 22a by injection molding, and mounting pins 23 are upright from the inner bottom surface 22a of the bottom surface portion 22 to the engaging surface 25 to be engaged with the grip case 3 in a vertical direction, and the housing 2 is engaged with the base 5 shown in FIG. 5 by screwing tips of the mounting pins 23.

Further, the housing 2 of the conventional ultrasonic probe having such structure is an injection-molded article, and the housing 2 is manufactured by providing a gate (an inlet of molten resin) G at one place of the peripheral portion 21 which is thick of the housing 2 in the longitudinal direction (or may be in the short axis direction) as shown in FIG. 6, and flowing molten resin F₀ from the gate G into a mold.

During injection molding, molten resin F₀ flowing from the gate G spreads like a fan into a cavity of the injection mold as molten resins F_{1,2,3} shown in FIG. 6. Here, flow speeds of the molten resins F_{1,2,3} in the cavity of the injection mold are different by the thickness of the housing 2 where the molten resin passes through (corresponding to the cross sectional area of flow path in the cavity).

In particular, the housing 2 of the ultrasonic probe, as shown in FIG. 6, thickness t₃ of the bottom surface portion 22 to be in contact with a body surface of a patient for ultrasonic transmission during ultrasonic diagnosis, is relatively thin and uniform while thickness t₁ from an outer circumferential edge of the bottom surface portion 22 to the peripheral portion 21 is relatively thick in order to attain a predetermined mechanical strength to the housing 2.

For this reason, flow speed of molten resin flowing in the cavity of the injection mold is that flow speed of the molten resin F₁ in the cavity, which forms the peripheral portion 21 is faster than flow speeds of the molten resins F₂, F₃ in the cavity, which form the bottom surface portion 22.

Therefore, before the molten resins F₂, F₃ in the cavity, which form the bottom surface portion 22, reaches the peripheral portion 21 opposite to the gate G, the molten resin F₁ flowing in from the gate G flows through the peripheral portion 21 which is thick and goes ahead, so that the molten resin F₁ flows pushing back the molten resins F₂, F₃ upwards.

In result, the molten resins F₂, F₃ which have flowed in the cavity to form the bottom surface portion 22 of the housing 2, collide the molten resin F₁ which has flowed in the cavity to form the peripheral portion 21 at the bottom surface portion 22 which is thin, and then are pushed upwards, which causes defect of injection molding, that is “weld line” on the outer surface of the bottom surface portion 22 which is thin.

Here, “weld line” is, as shown in FIG. 7, a V-shaped groove-like linear mark (defect) occurs between the molten resin and the surface of the cavity of the injection mold where two or more molten resins meet in the cavity of the injection mold.

### SUMMARY

### Technical Problem

When such weld line occurs on the outer surface of the bottom surface portion which is thin of the housing, in addition to its thin thickness, mechanical strength of the portion where the weld line occurred generally decreases, compared to the other portions. For this reason, when shock, e.g., due to fall is applied to the ultrasonic probe, the portion (weld line) may be easily cracked. In result, in a case of the housing being broken, it is a problem in that because acoustic propagation medium, e.g., oil sealed therein flows out of the housing, the ultrasonic probe cannot be used.

### Solution to Problem

The present invention has been made to solve the problem of the conventional ultrasonic probe. An ultrasonic probe of one embodiment of the present invention comprises: an ultrasonic transmission and reception unit provided inside a housing; an acoustic transmission medium sealed in the housing; and a drive device for oscillating the ultrasonic transmission and reception unit. In the ultrasonic probe, the housing is an injection-molded article by plastic material, and has a container shape with an opening in one direction. The container includes a bottom surface portion through which ultrasonic waves transmit and a peripheral portion engaged with a probe body. The housing is injection-molded by providing the peripheral portion having a thickness thicker than a thickness of the bottom surface portion, providing a thin portion at a part of the peripheral portion, and providing a gate at a part of the peripheral portion apart from the thin portion.

Further, in the ultrasonic probe, the thin portion is a vertical groove formed at an inner surface of the peripheral portion, parallel to a direction of mold release.

Furthermore, in the ultrasonic probe, the thin portion is a vertical groove formed at an outer surface of the peripheral portion in a direction of mold release.

Furthermore, in the ultrasonic probe of the present invention, a drive device is disposed for oscillating the ultrasonic transmission and reception unit in a short axis direction.

In an ultrasonic probe of the present invention, a drive device is disposed for oscillating the ultrasonic transmission and reception unit in a longitudinal direction to reciprocate.

An injection molding method for the afore described ultrasonic probe comprising: proving a gate at a part of the peripheral portion, so that a weld line occurs at an outer surface of the peripheral portion which is thick opposing to the peripheral portion where the gate is provided.

### Effects of the Invention

According to the present invention, it avoids occurrence of the weld line on the outer surface of the thin bottom surface portion of the housing and thus prevents damage of the housing when shock is applied to the ultrasonic probe.

### BRIEF DESCRIPTION OF DRAWINGS

FIG. 1 is a perspective view showing a housing of an ultrasonic probe molded by an injection molding method for an ultrasonic probe of the present invention, seen from inside of a bottom surface of the housing.

FIG. 2 is a perspective view showing the housing shown in FIG. 1, taken along a central line in a longitudinal direction, seen from a body contacting surface thereof.

FIG. 3 is a partially enlarged view of a part of the housing, pointed by an arrow A shown in FIG. 1.

FIG. 4 is a front view of a convention ultrasonic probe.

FIG. 5 is a vertical cross sectional view of the conventional ultrasonic probe, pointed by an arrow B shown in FIG. 4.

FIG. 6 is a perspective view showing a housing of the conventional ultrasonic probe shown in FIG. 4, taken along a central line in a longitudinal direction, seen from a body contacting surface thereof.

FIG. 7 is a schematic view showing a condition of injection molding that weld line occurs on a part where molten resins meet in the cavity of the mold.

### EMBODIMENTS OF THE INVENTION

Embodiments of an ultrasonic probe and an injection molding method for the same will be described with accompanying drawings in the following.

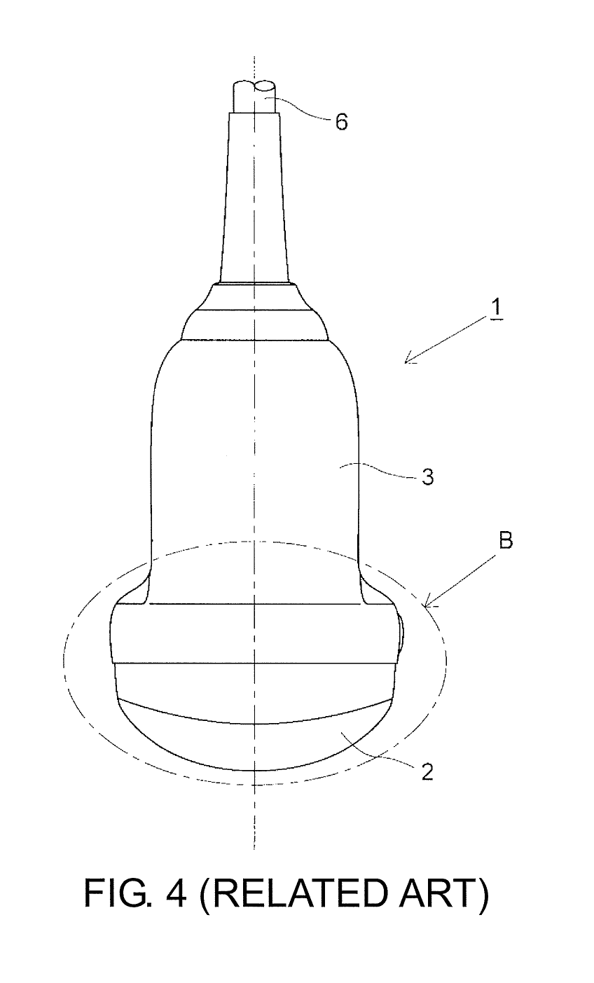

An ultrasonic probe to which a housing molded by the injection molding method of the present invention is applied, as well as the conventional ultrasonic probe shown in FIGS. 4-6, comprises: a housing 2, being injection molded by polyolefin thermoplastic material and having an opening 26 formed in one direction; a grip case 3, engaging with the housing 2; and a power supply cable 6 for supplying power to a drive device for oscillating a probe body 4. And, the probe body 4 constituting an ultrasonic transmission and reception unit including a piezoelectric element group and others is disposed at a base 5 provided inside the housing 2, and an acoustic propagation medium L, e.g., oil is also contained and sealed in the housing 2, and a body contacting surface of the housing 2 is brought into contact with a body surface of a patient and the probe body 4 is oscillated in a short-axial direction of the piezoelectric element group, so as to take in three dimensional data of a detected object.

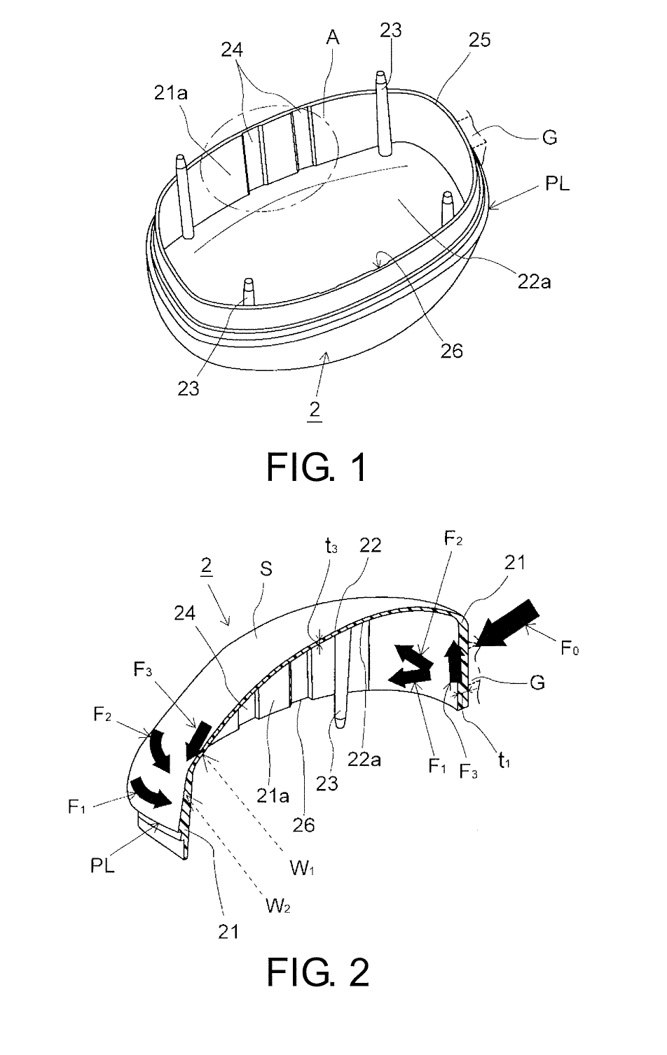

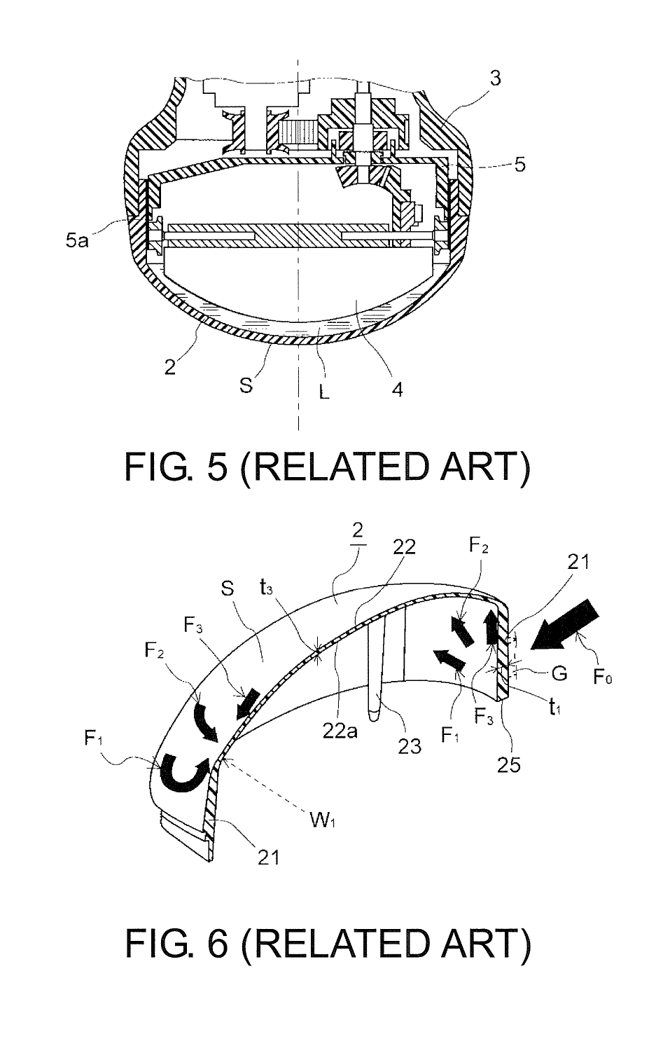

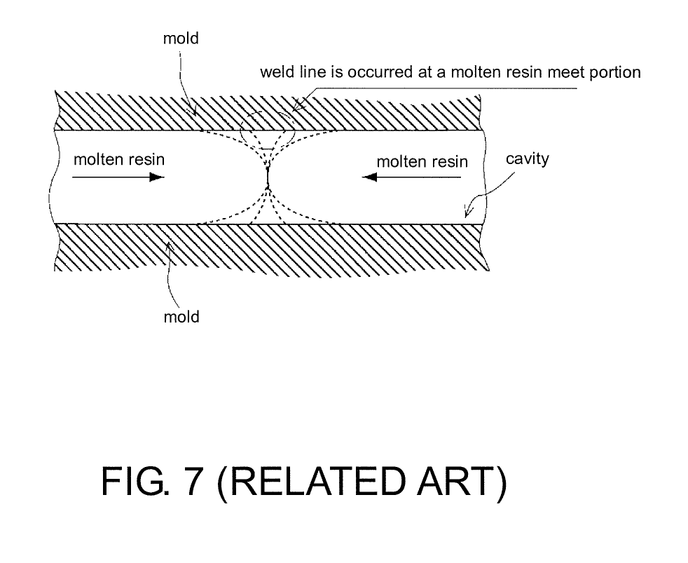

As shown in FIG. 1, the housing 2 has an outer curved surface shape like a rugby ball cut into two along a center line in a longitudinal direction, and the housing 2 comprises: a peripheral portion 21 which is thick and extends in a vertical direction from an engaging surface 25 to be engaged with the grip case 3; and a bottom surface portion 22 which is thin and has curved surface shape so as to cover the peripheral portion 21. Then, mounting pins 23, for example, four, are made integrated with an inner bottom surface 22a by injection molding and mounting pins 23 are upright from the inner bottom surface 22a of the bottom surface portion 22 to an end surface of the housing 2, i.e., the engaging surface 25 in a vertical direction, and the housing 2 is fixed at the base 5 shown in FIG. 5 by screwing tips of the mounting pins 23.

Further, the housing 2 of the ultrasonic probe having such structure is made by providing a gate (an inlet of molten resin) G having a flow passage cross sectional area corresponding to amount of molten resin of the housing 2 to be molded by injection molding, along a parting line (a mold parting line) PL at one place of the peripheral portion 21 which is thick of the housing 2 in the longitudinal direction (or may be in the short axis direction) as shown in FIG. 2, and flowing molten resin F₀ into a cavity of an injection mold from the gate G, passing through an injection nozzle, a sprue, a runner and others of injection molding apparatus which is not shown.

Here, for the gate G, any type of gates, e.g., a side gate, a film gate, a ring gate, a pin gate, and a point gate can be used, if it fits to the injection molding of this kind of injection-molded article.

Further, as molten resin to be used in injection molding of the ultrasonic probe of the present invention, for example, a plastic material that is a polyolefin thermoplastic resin is used to melt to temperature of around 280° C.-300° C.

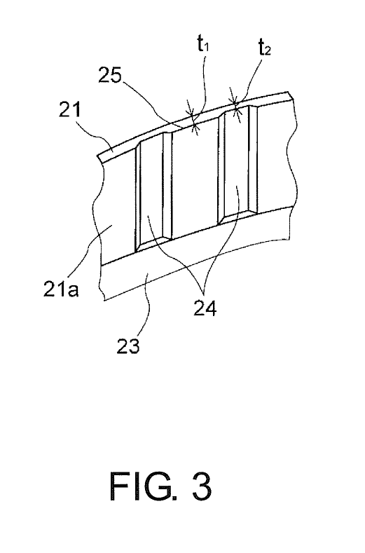

In the embodiment of the injection molding method for the ultrasonic probe of the present invention, as shown in FIG. 2 and FIG. 3, a plurality of vertical grooves 24 each having a predetermined width are formed at an inner wall surface 21 a of the peripheral portion 21 of the housing 2 in a direction perpendicular to PL (a mold parting line: a parting line) (a direction of mold release) between the gate G and the peripheral portion 21 of the housing opposing to the gate G, in order to prevent weld line from occurring at the bottom surface portion 22 which is thin of the housing, which is the problem of the injection molding of the conventional ultrasonic probe. Here, inner side surfaces of these vertical grooves 24 may be parallel to the direction of mold release or may be provided with a draft angle for easy mold release.

As the vertical grooves 24 are funned at the inner wall surface 21a of the peripheral portion 21, as shown in FIG. 3, portions having thin thicknesses t₂ relative to the general thickness t₁ of the inner wall surface 21a are formed at the inner wall surface 21a. Since cross sectional area of flow path for molten resin F₁ flowing therein is narrowed at the thin portions of thickness t₂, flow speed of the molten resin F₁ is lowered due to flow resistance at the thin portions, while flow speed of the molten resin F₃ flowing in the cavity of the mold, which forms the bottom surface portion 22 having thickness t₃ of the housing 2, becomes faster.

That is, molten resin F₀ flowing in from the gate G spreads like a fan into a cavity (a runner) of the injection mold as molten resins F_{1,2,3}. Here, flow speeds of the molten resins F_{1,2,3} in the cavity of the injection mold are different by the thickness of the housing 2 where they pass through (corresponding to the cross sectional area of flow path in the cavity).

However, in the injection molding method for the ultrasonic probe of the present invention, because plural vertical groves 24 are formed at the inner wall surface 21a of the peripheral portions 21, the molten resin F₁ which flows from the gate G through both sides of the peripheral portion 21 and reaches the end opposing to the gate G, is pushed back by the molten resins F_{2 and 3} which flow in the cavity to form the bottom surface portion 22 which is thin and has curved surface shape, thereby controlling the behavior of the flowing direction of the molten resin. For this reason, conventionally, weld line W₁ occurred near the portion shown by a dotted line (the bottom surface portion 22 where the thickness t₃ is thin) is shifted to weld line W₂ occurring at the peripheral portion where the thickness is thick and mechanical strength is high.

Therefore, since weld line W₂ occurs at the peripheral portion where the thickness is thick and the mechanical strength is high, not at the bottom surface portion 22 which is thin and has curved surface shape of the housing, even if the ultrasonic probe is dropped during operation and shock is applied to the housing 2, the housing 2 will not be broken from the weld line W₂.

Further, in the ultrasonic probe of the present invention, the plural vertical grooves 24 are formed at the inner wall surface 21a of the housing 2 at interval, and thus, as shown in FIG. 5, adhesive strength of adhesion 5a applied over whole circumference between the inner surface 22a of the housing 2 and the outer surface of the base 5 increases to prevent acoustic propagation medium, e.g., oil from leaking from the inside of the housing 2.

It is noted that after the injection-molded article, i.e., the housing 2 is released from the mold, during a deflashing process, the gate G is removed from the outer peripheral surface of the peripheral portion 21 of the housing 2, i.e., the injection-molded article, by an appropriate tool.

It is noted that in the present embodiment, the vertical grooves 24 are formed at the inner wall surface 21a of the peripheral potion 21, but the vertical grooves 24 may be provided at an outer wall surface of the peripheral portion 21 of the housing if its structure does not affect the appearance design.

Further, in the present embodiment, the bottom surface portion 22 of the housing 2 is in curved surface shape and whole structure shaped like a rugby ball cut into two along a center line in a longitudinal direction has been described, but the present invention can be applied to the structure like a linear oscillation (reciprocating motion) type ultrasonic probe in which the bottom surface portion (ultrasonic wave transmitting surface) of the housing 2 has a thin planar shape and the peripheral portion thereof has a shape of a thick box.

### DESCRIPTION OF THE REFERENCE NUMERALS

1 ultrasonic probe
2 housing
3 snap case
4 ultrasonic transmission and reception unit (probe body)
5 base
6 power supply cable
21 peripheral portion
22 bottom surface portion
23 mounting pin
24 vertical groove
25 housing end surface (engaging surface)
26 opening
W weld line
G gate
F flow of molten resin
S body surface contacting surface
L acoustic propagation medium
PL mold line parting line (parting line)

1. An ultrasonic probe, comprising:
an ultrasonic transmission and reception unit, being provided inside a housing;
an acoustic transmission medium, being sealed in the housing; and
a drive device for oscillating the ultrasonic transmission and reception unit;
wherein the housing being an injection-molded article by plastic material, and having a container shape with an opening in one direction,
the container shape includes: a bottom surface portion through which ultrasonic waves transmit, and a peripheral portion being engaged with a probe body;
the housing being injection-molded by providing the peripheral portion having a thickness thicker than a thickness of the bottom surface portion, providing a thin portion at a part of the peripheral portion; and providing a gate at a part of the peripheral portion apart from the thin portion;
the thin portion being formed along a vertical direction perpendicular to a circumference direction of the peripheral portion.

2. An ultrasonic probe according to claim 1, wherein
the thin portion is a vertical groove formed at an inner surface of the peripheral portion, parallel to a direction of mold release.

3. An ultrasonic probe according to claim 1, wherein
the thin portion is a vertical groove formed at an outer surface of the peripheral portion, parallel to a direction of mold release.

4. An ultrasonic probe according to claim 1, wherein
the drive device is disposed for oscillating the ultrasonic transmission and reception unit in a short axis direction of the housing.

5. An ultrasonic probe according to claim 1, wherein
the drive device is disposed for oscillating the ultrasonic transmission and reception unit in a longitudinal direction of the housing to reciprocate.

6. An injection molding method for an ultrasonic probe according to claim 1, comprising:
proving the gate at a part of the peripheral portion, so that a weld line occurs at an outer surface of the peripheral portion which is thick opposing to the peripheral portion where the gate is provided.

7. An injection molding method for an ultrasonic probe according to claim 2, comprising:
proving the gate at a part of the peripheral portion, so that a weld line occurs at an outer surface of the peripheral portion which is thick opposing to the peripheral portion where the gate is provided.

8. An injection molding method for an ultrasonic probe according to claim 3, comprising:
proving the gate at a part of the peripheral portion, so that a weld line occurs at an outer surface of the peripheral portion which is thick opposing to the peripheral portion where the gate is provided.

9. An injection molding method for an ultrasonic probe according to claim 4, comprising:
proving the gate at a part of the peripheral portion, so that a weld line occurs at an outer surface of the peripheral portion which is thick opposing to the peripheral portion where the gate is provided.

10. An injection molding method for an ultrasonic probe according to claim 5, comprising:
proving the gate at a part of the peripheral portion, so that a weld line occurs at an outer surface of the peripheral portion which is thick opposing to the peripheral portion where the gate is provided.
